(19) United States
(12) Patent Application Publication
(10) Pub. No.: US 2010/0075979 A1
(43) Pub. Date: Mar. 25, 2010

(54) PYRIDINYLPIPERAZIN DERIVATIVES USEFUL AS MODULATORS OF DOPAMINE D3 RECEPTORS

(76) Inventors: Luca Gobbi, Muttenz (CH); Georg Jaeschke, Basel (CH); Rosa Maria Rodriguez Sarmiento, Basel (CH); Lucinda Steward, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/560,463

(22) Filed: Sep. 16, 2009

(30) Foreign Application Priority Data
Sep. 23, 2008 (EP) 08164898.2

(51) Int. Cl. A61K 31/496; C07D 401/04

(52) U.S. Cl. 514/253.01; 544/360

(57) ABSTRACT

The present invention provides compounds of provides formula I, having affinity and selectivity for the dopamine D3 receptors, their manufacture, pharmaceutical compositions containing them and their use for the therapeutic and/or prophylactic treatment of cognitive disorders.

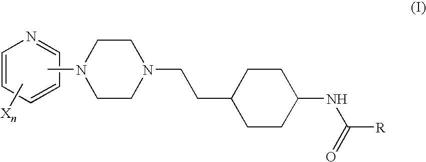

### PRIORITY TO RELATED APPLICATION(S)

This application claims the benefit of European Patent Application No. 08164898.2, filed Sep. 23, 2008, which is hereby incorporated by reference in its entirety.

### BACKGROUND INFORMATION

Dopamine, a major catecholamine neurotransmitter, is involved in the regulation of a variety of functions which include emotion, cognition, motor functions, and positive reinforcement, (Purves, D. et al. (2004) Neuroscience. Sinauer, third edition, Sunderland, Mass.). The biological activities of dopamine are mediated through G protein-coupled receptors (GPCRs) and in human, five different dopamine receptors D₁-D₅ have been identified, where the D₂-like receptors (D₂, D₃ and D₄) couple to the G-protein G_{αI} (Missale, C. et al. (1998) Dopamine receptors: from structure to function. Physiol. Rev. 78, 189-225). The D₃ dopamine receptor is most highly expressed in the nucleus accumbens (Gurevich, E. V., Joyce, J. N. (1999)

Distribution of dopamine D3 receptor expressing neurons in the human forebrain: comparison with D2 receptor expressing neurons. Neuropsychopharmacology 20, 60-80), and is proposed to modulate the mesolimbic pathway consisting of neuronal projections from the ventral tegmental area, hippocampus and amygdala to the nucleus accumbens, which projects to the prefrontal and cingulate cortices as well as various thalamic nuclei. The limbic circuit is thought to be important for emotional behavior and thus D₃ receptor antagonists are proposed to modulate psychotic symptoms such as hallucinations, delusions and thought disorder (Joyce, J. N. and Millan,

M. J., (2005) Dopamine D3 receptor antagonists as therapeutic agents. Drug Discovery Today, 1 July, Vol. 10, No. 13, 917-25), while these antagonists spare the D₂ modulated striatal extrapyramidal system (associated with EPS induction). In addition, it has been reported that drug naive schizophrenic patients show altered levels of D₃ receptor expression (Gurevich, E. V. et al. (1997) Mesolimbic dopamine D3 receptors and use of antipsychotics in patients with schizophrenia. A postmortem study. Arch. Gen. Psychiatry 54, 225-232) and dopamine release (Laruelle, M. (2000) Imaging dopamine dysregulation in schizophrenia: implication for treatment. Presented at Workshop Schizophr.: Pathol. Bases and Mech. Antipsychotic Action, Chicago), indicating that a disturbed homeostasis of dopamine plays an important role in the etiology of schizophrenic symptoms.

### SUMMARY OF THE INVENTION

The present invention relates to compounds of formula I,

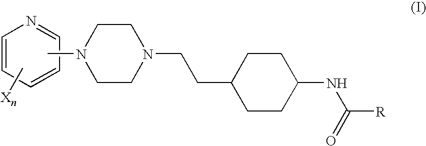

wherein:
X is independently halogen, C₁₋₆-alkyl, C₁₋₆-haloalkyl or C₁₋₆-alkoxy;
n is 1 or 2;
R is C₁₋₆-alkyl, wherein C₁₋₆-alkyl is optionally substituted by —CONH₂, C₁₋₆-alkoxy, or one 3 to 6 membered monocyclic cycloalkyl; or
   C₁₋₆-alkoxy;
      as well as pharmaceutically acceptable salts thereof.

Compounds of formula I have affinity for dopamine D3 receptors and thus are useful in the treatment of conditions wherein modulation, especially antagonism/inhibition, of D3 receptors is beneficial, e.g. to treat drug dependency or as antipsychotic agents.

### DETAILED DESCRIPTION OF THE INVENTION

Compounds of formula I and their pharmaceutically acceptable salts are useful in the treatment of all aspects of drug dependency, including drug intake, relapse to drug-seeking behaviour following abstinence and withdrawal symptoms from drugs of abuse such as alcohol, cocaine, opiates, nicotine, benzodiazepines and inhibition of tolerance induced by opioids, as well as for the treatment of drug craving. They also are useful as an antipsychotic agent for example in the treatment of schizophrenia, schizo-affective disorders, schizophreniform diseases, psychotic depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion), anxiety disorders (which includes generalized anxiety and social anxiety disorder), mania, acute mania, paranoid and delusional disorders. The compounds are also useful for the treatment of a family of related disorders referred to as somatoform disorders, as well as for the treatment of premature ejaculation. The compounds are further useful for the treatment of attention-deficit hyperactivity disorder (ADHD), addiction (smoking cessation, cocaine and others) and obsessive compulsive disorder (OCD).

Compounds of formula I can form acid addition salts with acids, such as conventional pharmaceutically acceptable acids, for example hydrochloride, hydrobromide, phosphate, acetate, fumarate, maleate, salicylate, sulphate, pyruvate, citrate, lactate, mandelate, tartarate, and methanesulphonate. Preferred are the hydrochloride salts. Also solvates and hydrates of compounds of formula I and their salts form part of the present invention.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbens or eluant). The invention embraces all of these forms.

It will be appreciated, that the compounds of formula I in this invention can be derivatized at functional groups to provide derivatives which are capable of conversion back to the parent compound in vivo. Physiologically acceptable and metabolically labile derivatives, which are capable of producing the parent compounds of formula I in vivo are also within the scope of this invention.

As used herein, the term “C₁₋₆-alkyl” denotes monovalent linear or branched saturated hydrocarbon moiety, consisting solely of carbon and hydrogen atoms, having from 1 to 6 carbon atoms, for example, methyl, ethyl, propyl, isopropyl, n-butyl, iso-butyl, sec-butyl, tert-butyl and the like. Preferred alkyl groups are groups with 1, 2, 3 or 4 carbon atoms. Most preferred alkyl groups are methyl and ethyl.

The term “halogen” denotes chlorine (chloro, Cl), iodine (iodo, I), fluorine (fluoro, F) and bromine (bromo, Br). Preferred halogen are fluoro, chloro and bromo, more preferred are fluoro and chloro, most preferred is fluoro.

The term “C₁₋₆-alkoxy” denotes a group —O—R′ wherein R′ is C₁₋₆-alkyl as defined above. Preferred C₁₋₆-alkoxy is methoxy.

The term “C₁₋₆-haloalkyl” denotes an alkyl group as defined above wherein at least one of the hydrogen atoms of the alkyl group is replaced by a halogen atom, preferably fluoro or chloro, most preferably fluoro. Examples of haloalkyl include but are not limited to methyl, ethyl, propyl, isopropyl, isobutyl, sec-butyl, tert-butyl, pentyl or n-hexyl wherein one or more hydrogen atoms are replaced by Cl, F, Br or I atom(s), as well as those haloalkyl groups specifically illustrated by the examples herein below. Among the preferred haloalkyl groups are monofluoro-, difluoro- or trifluoro-methyl, -ethyl or -propyl, for example 3,3,3-trifluoropropyl, 2-fluoroethyl, 2,2,2-trifluoroethyl, fluoromethyl, trifluoromethyl.

The phrase “3 to 6 membered monocyclic cycloalkyl” refers to a monovalent saturated monocyclic hydrocarbon radical of 3 to 6 ring carbon atoms. Examples are cyclopropyl, cyclobutanyl, cyclopentyl or cyclohexyl. Preferred examples are cyclopropyl, cyclopentyl and cyclohexyl.

The phrase “4 to 6 membered monocyclic heterocycloalkyl” refers to a monovalent saturated 4- to 6-membered monocyclic ring system containing one, two or three ring heteroatoms selected from N, O and S, the remaining ring atoms being carbon atoms. 5- or 6-membered monocyclic heterocycloalkyls are preferred. “Heterocycloalkyl” can be unsubstituted or substituted as described herein. Preferred is tetrahydropyranyl.

“Pharmaceutically acceptable,” such as pharmaceutically acceptable carrier, excipient, etc., means pharmacologically acceptable and substantially non-toxic to the subject to which the particular compound is administered.

The terms “pharmaceutically acceptable salt” or “pharmaceutically acceptable acid addition salt” embrace salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

When indicating the number of substituents, the term “one or more” means from one substituent to the highest possible number of substitution, i.e. replacement of one hydrogen up to replacement of all hydrogens by substituents. Thereby, one, two or three substituents are preferred.

In detail, the present invention provides compounds of formula I,

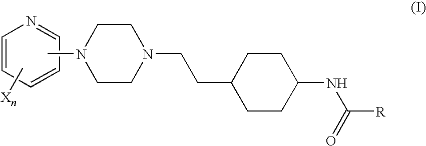

wherein:
X is independently halogen, C₁₋₆-alkyl, C₁₋₆-haloalkyl or C₁₋₆-alkoxy;
n is 1 or 2;
R is C₁₋₆-alkyl, wherein C₁₋₆-alkyl is optionally substituted by —CONH₂, C₁₋₆-alkoxy, or one 3 to 6 membered monocyclic cycloalkyl; or
   C₁₋₆-alkoxy;
      as well as pharmaceutically acceptable salts thereof.

In a preferred embodiment the present invention provides a compound of formula I′,

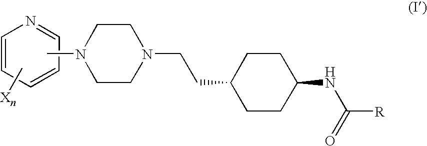

wherein R, X and n are defined as given above.

Preference is given to compounds of formulae Ia or Ia′:

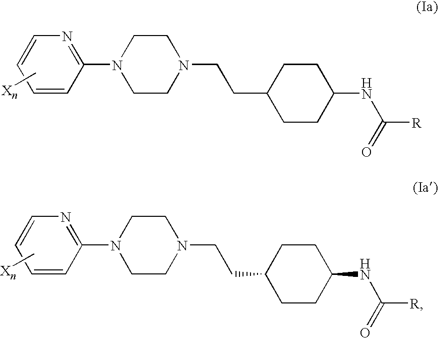

wherein R, X and n are defined as given above.

Preference is given to compounds of formulae Ib or Ib′:

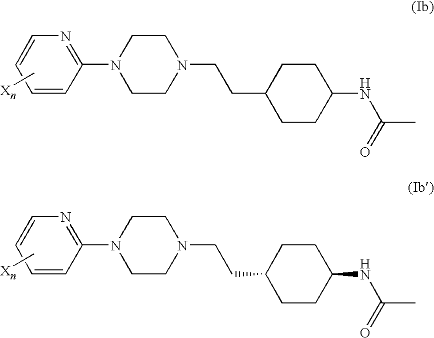

wherein R, X and n are defined as given above.

Preference is given to compounds of formulae Ib or Ib′, wherein X is independently fluorine, chlorine, —CF₃ or —OCH₃; and n is 1 or 2.

Special preference is given to a compound of formula (I′) selected from the group consisting of:
N-(trans-4-{2-[4-(3-Chloro-5-trifluoromethyl-pyridin-2-yl)-piperazin-1-yl]-ethyl}-cyclohexyl)-acetamide;
N-(trans-4-{2-[4-(3-Chloro-5-trifluoromethyl-pyridin-2-yl)-piperazin-1-yl]-ethyl}-cyclohexyl)-3-methoxy-propionamide;
N-(trans-4-{2-[4-(3-Chloro-5-trifluoromethyl-pyridin-2-yl)-piperazin-1-yl]-ethyl}-cyclohexyl)-propionamide;
N-(trans-4-{2-[4-(3-Chloro-5-trifluoromethyl-pyridin-2-yl)-piperazin-1-yl]-ethyl}-cyclohexyl)-2-cyclopropyl-acetamide;
N-(trans-4-{2-[4-(3-Chloro-pyridin-2-yl)-piperazin-1-yl]-ethyl}-cyclohexyl)-acetamide;
N-(trans-4-{2-[4-(3,5-Dichloro-pyridin-2-yl)-piperazin-1-yl]-ethyl}-cyclohexyl)-acetamide;
N-(trans-4-{2-[4-(6-Trifluoromethyl-pyridin-3-yl)-piperazin-1-yl]-ethyl}-cyclohexyl)-acetamide;
N-(trans-4-{2-[4-(3-Chloro-5-trifluoromethyl-pyridin-2-yl)-piperazin-1-yl]-ethyl}-cyclohexyl)-malonamide;
N-(trans-4-{2-[4-(3-Methoxy-pyridin-2-yl)-piperazin-1-yl]-ethyl}-cyclohexyl)-acetamide; and
N-(trans-4-{2-[4-(2,3-Dichloro-pyridin-4-yl)-piperazin-1-yl]-ethyl}-cyclohexyl)-acetamide.

In one embodiment, the invention provides compounds of formulae I, I′, Ia, Ia′, Ib, Ib′ wherein X is independently halogen, C₁₋₆-alkyl, C₁₋₆-haloalkyl or C₁₋₆-alkoxy.

In one embodiment, the invention provides compounds of formulae I, I′, Ia, Ia′, Ib, Ib′ wherein X is halogen.

In one embodiment, the invention provides compounds of formulae I, I′, Ia, Ia′, Ib, Ib′ wherein X is fluorine.

In one embodiment, the invention provides compounds of formulae I, I′, Ia, Ia′, Ib, Ib′ wherein X is chlorine.

In one embodiment, the invention provides compounds of formulae I, I′, Ia, Ia′, Ib, Ib′ wherein X is C₁₋₆-alkyl.

In one embodiment, the invention provides compounds of formulae I, I′, Ia, Ia′, Ib, Ib′ wherein X is C₁₋₆-haloalkyl.

In one embodiment, the invention provides compounds of formulae I, I′, Ia, Ia′, Ib, Ib′ wherein X is —CF₃.

In one embodiment, the invention provides compounds of formulae I, I′, Ia, Ia′, Ib, Ib′ wherein X is C₁₋₆-alkoxy.

In one embodiment, the invention provides compounds of formulae I, I′, Ia, Ia′, Ib, Ib′ wherein X is —OCH₃.

In one embodiment, the invention provides compounds of formulae I, I′, Ia, Ia′, Ib, Ib′ wherein X is independently chlorine, fluorine, —CF₃ or —OCH₃.

In one embodiment, the invention provides compounds of formulae I, I′, Ia, Ia′, Ib, Ib′ wherein n is 1 or 2.

In one embodiment, the invention provides compounds of formulae I, I′, Ia, Ia′, Ib, Ib′ wherein n is 1.

In one embodiment, the invention provides compounds of formulae I, I′, Ia, Ia′, Ib, Ib′ wherein n is 2.

In one embodiment, the invention provides compounds of formulae I, I′, Ia or Ia′ wherein R is
C₁₋₆-alkyl, wherein C₁₋₆-alkyl is optionally substituted by —CONH₂ or one 3 to 6 membered monocyclic cycloalkyl; or
C₁₋₆-alkoxy.

In one embodiment, the invention provides compounds of formulae I, I′, Ia or Ia′ wherein R is methyl, methyl substituted by —CONH₂, methyl substituted by cyclopropyl, ethyl or ethyl substituted by —OCH₃.

In one embodiment, the invention provides compounds of formulae I, I′, Ia or Ia′ wherein R is C₁₋₆-alkyl.

In one embodiment, the invention relates to compounds of formulae I, I′, Ia or Ia′ wherein R is methyl.

In one embodiment, the invention provides compounds of formulae I, I′, Ia or Ia′ wherein R is ethyl.

In one embodiment, the invention provides compounds of formulae I, I′, Ia or Ia′ wherein R is C₁₋₆-alkyl substituted by —CONH₂.

In one embodiment, the invention provides compounds of formulae I, I′, Ia or Ia′ wherein R is methyl substituted by —CONH₂.

In one embodiment, the invention provides compounds of formulae I, I′, Ia or Ia′ wherein R is C₁₋₆-alkyl substituted by 3 to 6 membered monocyclic cycloalkyl.

In one embodiment, the invention provides compounds of formulae I, I′, Ia or Ia′ wherein R is methyl substituted by cyclopropyl.

In one embodiment, the invention provides compounds of formulae I, I′, Ia or Ia′ wherein R is C₁₋₆-alkoxy.

In one embodiment, the invention provides compounds of formulae I, Ia or Ia′ wherein R is ethyl-OCH₃.

A further aspect of the present invention provides a method for the treatment and/or the prevention of cognitive disorders, drug addiction, depression, anxiety, drug dependence, dementias, memory impairment, psychotic disorders comprising schizophrenia, schizoaffective disorders, bipolar disease, mania, psychotic depression, psychoses comprising paranoia and delusions, attention-deficit hyperactivity disorder, addiction and obsessive compulsive disorder which comprises administering a therapeutically effective amount of a compound of formulae I, I′, Ia, Ia′, Ib, or Ib′.

A further aspect of the present invention provides pharmaceutical compositions containing a therapeutically effective amount of compounds of formulae I, I′, Ia, Ia′, Ib, Ib′ or a pharmaceutically acceptable salt thereof and a pharmaceutically acceptable carrier.

A further aspect of the present invention provides a method for the treatment of schizophrenia, cognitive disorders and drug addiction which comprises administering a compound of formula I, I′, Ia, Ia′, Ib, or Ib′ or a pharmaceutically acceptable salt thereof.

A further aspect of the present invention provides the process for the manufacture of compounds of formulae I, I′, Ia, Ia′, Ib, Ib′ as defined above.

A further aspect of the present invention provides a method for the treatment or prevention of diseases related to the D3 receptor which comprises administering a compound of formula I, I′, Ia, Ia′, Ib, or Ib′ or a pharmaceutically acceptable salt thereof.

A further aspect of the present invention provides a method for the therapeutic and/or prophylactic treatment of a disorder or condition mediated by the D3 receptor binding site, or that can be treated via modulation of the D3 receptor binding site, particularly for the therapeutic and/or prophylactic treatment of cognitive disorders, drug addiction, depression, anxiety, drug dependence, dementias, memory impairment, psychotic disorders comprising schizophrenia, schizoaffective disorders, bipolar disease, mania, psychotic depression, psychoses comprising paranoia and delusions, attention-deficit hyperactivity disorder, addiction and obsessive compulsive disorder, which method comprises administering a compound formulae I, I′, Ia, Ia′, Ib, Ib′ to a human being or animal.

The preparation of compounds of formula I of the present invention can be carried out in sequential or convergent synthetic routes. Syntheses of the invention are shown in the following schemes. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. Starting materials are either commercially available or can be prepared by methods analogous to the methods given below, by methods described in references cited in the description or in the examples, or by methods known in the art.

A preferred embodiment of the process for preparing a compound of formula I,

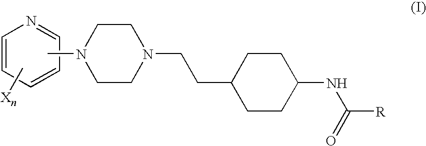

wherein R, X and n have meanings as given above,
comprises one of the following steps:
a) reductive amination of an aldehyde of formula (I-1) with a piperazine derivative of formula (I-2) in the presence of a reducing agent, and

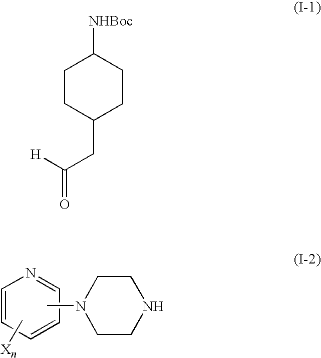

removing the Boc protecting group under acidic conditions to yield amine intermediate of formula (I-3)

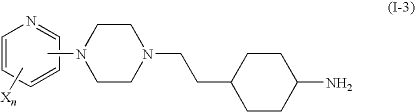

and
b) coupling of amine intermediate of formula (I-3) with a carboxylic acid R—COOH or acid chloride R—COCl to yield compound of formula I.

The ability of the compounds to bind to the D₃ receptors was determined using radioligand binding to cloned receptors selectively expressed in HEK-293 EBNA cells.

### Biological Data

### Membrane Preparation for Human D₃ Receptors

HEK-293 EBNA cells were transiently transfected with expression plasmids encoding for the human D₃ dopamine receptor. The cells were harvested 48 h post-transfection, washed three times with cold PBS and stored at −80° C. prior to use. The pellet was suspended in cold 50 mM

Tris-HCl buffer containing 10 mM EDTA (pH 7.4) and homogenized with a Polytron (Kinematica AG, Basel, Switzerland) for 20-30 sec at 12.000 rpm. After centrifugation at 48.000×g for 30 min at 4° C., the pellet was resuspended in cold 10 mM Tris-HCl buffer containing 0.1 mM EDTA (pH 7.4), homogenized, and centrifuged as above. This pellet was further resuspended in a smaller volume of ice cold 10 mM Tris-HCl buffer containing 0.1 mM EDTA (pH 7.4) and homogenized with a Polytron for 20-30 sec at 12.000 rpm. The protein content of this homogenate was determined with the Bio-Rad (Bradford) Protein Assay (Biorad Laboratories GmbH, München, Germany) according to the instructions of the manufacturer using gamma globulin as the standard.

This homogenate was stored at −80° C. in aliquots and thawed immediately prior to use.

### Radioligand Binding Assay Conditions

Aliquots of membrane preparations were thawed at RT, resuspended in assay buffer (50 mM Tris-HCl, 120 mM NaCl, 5 mM MgCl₂, 1 mM EDTA, 5 mM KCl, 1.5 mM CaCl₂, pH=7.4), homogenized with a Polytron for 20-30 sec at 12.000 rpm and adjusted to a final concentration of approximately 7.5 μg protein/well.

The binding affinity (Ki) of the compounds was determined using radioligand binding. Membranes were incubated in a total volume of 200 μl with a fixed concentration of radioligand (final concentration approximately 0.5 nM [³H]-spiperone) and ten concentrations of test compound in ranging between 10 μM-0.1 nM for 1 h at RT. At the end of the incubation, the reaction mixtures were filtered on to unifilter 96-well white microplates with bonded GF/C filters (Packard BioScience, Zurich, Switzerland; preincubated for 1 h in 0.1% polyethylenimine (PEI) in assay buffer) with a Filtermate 196 harvester (Packard BioScience) and washed 3 times with cold assay buffer. The nonspecific binding was determined with equally composed reaction mixtures in the presence of 10 μM unlabelled spiperone. Per well 45 μl of Microscint 40 (Perkin Elmer, Schwerzenbach, Switzerland) was added, plates for sealed, shaken for 20 min and counted for 3 min on a Topcount Microplate Scintillation Counter (Canberra Packard SA, Zürich, Switzerland) with quenching correction.

### Data Calculation

The CPM value for each duplicate of a concentration of competing compound was averaged (y1), then the % specific binding was calculated according to the equation (((y1−non-specific)/(total binding-non-specific))×100). Graphs were plotted with the % specific binding using XLfit, a curve fitting program that iteratively plots the data using Levenburg Marquardt algorithm. The single site competition analysis equation used was y=A+((B−A)/(1+((x/C)^{D}))), where y is the % specific binding, A is the minimum y, B is the maximum y, C is the IC₅₀, x is the log₁₀ of the concentration of the competing compound and D is the slope of the curve (the Hill Coefficient). From these curves the IC₅₀ (inhibition concentration at which 50% specific binding of the radioligand was displaced) and Hill coefficient were determined. The affinity constant (Ki) was calculated using the Cheng-Prusoff equation Ki=(IC₅₀/1+([L]/Kd), where [L] is the concentration of radioligand and Kd is the dissociation constant of the radioligand at the receptor as determined by the saturation isotherm.

The compounds of the present invention are potent modulators of the dopamine D₃ receptors as shown in the activity table hereinafter which gives the Ki values in μM for the dopamine D₃ receptors for some examples of the compounds of the present invention:

TABLE 1
acticity table: human Ki values of selected examples

| | | | Ki dopamine D3 |
|---|---|---|---|
| | | | receptor: Human |
| Ex. | Compound | Name | (D3) |
| 1 | 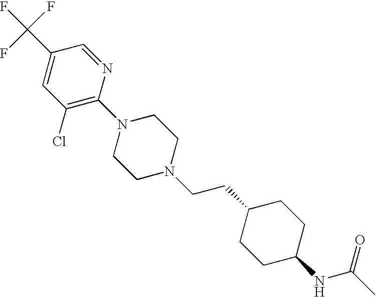 | N-(trans-4-{2-[4-(3,5- Dichloro-pyridin-2-yl)- piperazin-1-yl]-ethyl}- cyclohexyl)-acetamide | 0.00964 |
| 2 | 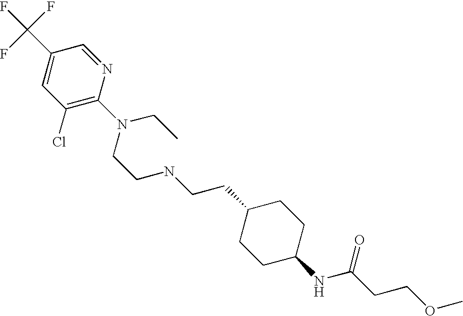 | N-(trans-4-{2-[4-(6- Trifluoromethyl-pyridin-3-yl)- piperazin-1-yl]-ethyl}- cyclohexyl)-acetamide | 0.005658 |
| 3 | 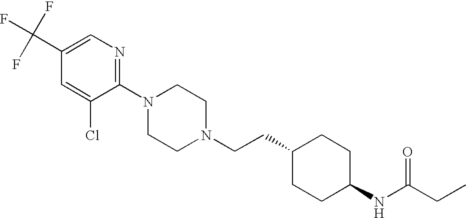 | N-(trans-4-{2-[4-(3-Chloro-5- trifluoromethyl-pyridin-2-yl)- piperazin-1-yl]-ethyl}- cyclohexyl)-malonamide | 0.016784 |
| 4 | 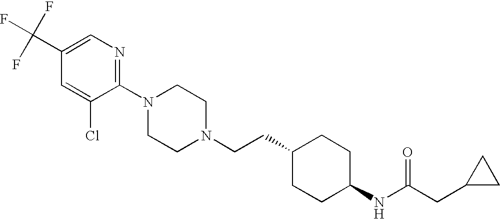 | N-(trans-4-{2-[4-(3-Methoxy- pyridin-2-yl)-piperazin-1-yl]- ethyl}-cyclohexyl)-acetamide | 0.010146 |
| 5 | 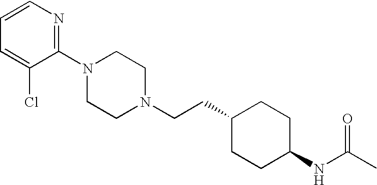 | N-(trans-4-{2-[4-(2,3- Dichloro-pyridin-4-yl)- piperazin-1-yl]-ethyl}- cyclohexyl)-acetamide | 0.001702 |
| 6 | 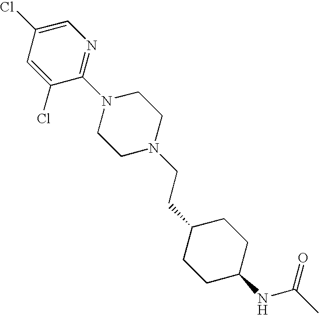 | N-(trans-4-{2-[4-(3,5- Dichloro-pyridin-2-yl)- piperazin-1-yl]-ethyl}- cyclohexyl)-acetamide | 0.00964 |
| 7 | 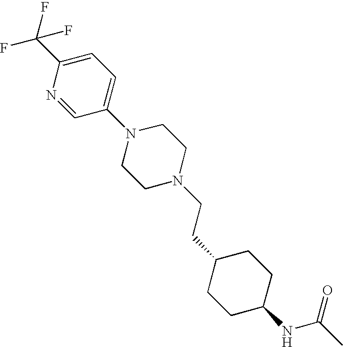 | N-(trans-4-{2-[4-(6- Trifluoromethyl-pyridin-3-yl)- piperazin-1-yl]-ethyl}- cyclohexyl)-acetamide | 0.005658 |
| 8 | 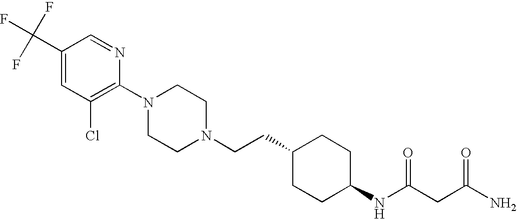 | N-(trans-4-{2-[4-(3-Chloro-5- trifluoromethyl-pyridin-2-yl)- piperazin-1-yl]-ethyl}- cyclohexyl)-malonamide | 0.016784 |
| 9 | 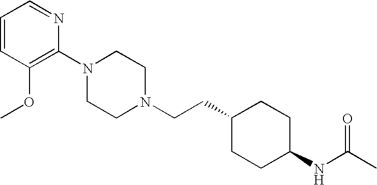 | N-(trans-4-{2-[4-(3-Methoxy- pyridin-2-yl)-piperazin-1-yl]- ethyl}-cyclohexyl)-acetamide | 0.010146 |
| 10 | 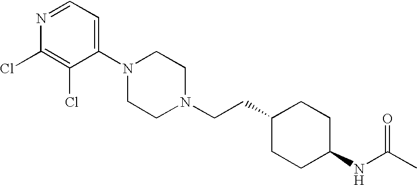 | N-(trans-4-{2-[4-(2,3- Dichloro-pyridin-4-yl)- piperazin-1-yl]-ethyl}- cyclohexyl)-acetamide | 0.001702 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acid or its salts and the like can be used, for example, as such as carriers for tablets, coated tablets, dragées and hard gelatine capsules. Suitable carriers for soft gelatine capsules are, for example, vegetable oils, waxes, fats, semi-solid and liquid polyols and the like; depending on the nature of the active substance no carriers are, however, usually required in the case of soft gelatine capsules.

Suitable carriers for the production of solutions and syrups are, for example, water, polyols, sucrose, invert sugar, glucose and the like. Adjuvants, such as alcohols, polyols, glycerol, vegetable oils and the like, can be used for aqueous injection solutions of water-soluble salts of compounds of formula I, but as a rule are not necessary. Suitable carriers for suppositories are, for example, natural or hardened oils, waxes, fats, semi-liquid or liquid polyols and the like.

In addition, the pharmaceutical compositions can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The present invention also provides a process for the manufacture of pharmaceutical compositions. Such process comprises bringing the compound of formula I and/or pharmaceutically acceptable acid addition salt thereof and, fir desired, one or more other therapeutically valuable substances into a galenical administration form together with one or more therapeutically inert carriers.

The dosage at which compounds of the invention can be administered can vary within wide limits and will, of course, be fitted to the individual requirements in each particular case. In general, the effective dosage for oral or parenteral administration is between 0.01-20 mg/kg/day, with a dosage of 0.1-10 mg/kg/day being preferred for all of the indications described. The daily dosage for an adult human being weighing 70 kg accordingly lies between 0.7-1400 mg per day, preferably between 7 and 700 mg per day.

### Synthesis

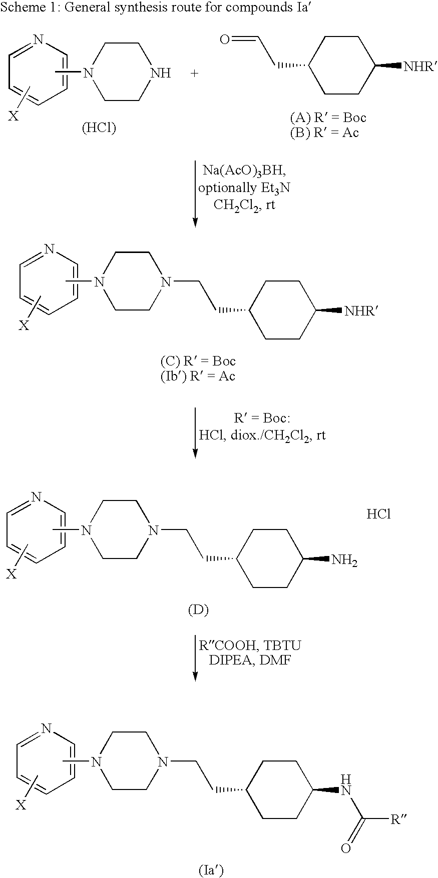

The starting materials are commercially available or the synthesis is described in the literature.

Compound (E3) can be prepared as shown hereinafter in Scheme 2.

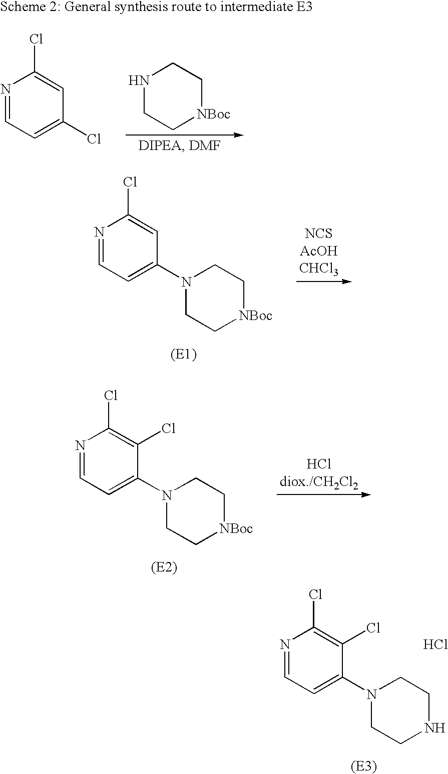

### Experimental Part

The following examples are provided to further elucidate the invention.

### Example 1

### N-(trans-4-{2-[4-(3-Chloro-5-trifluoromethyl-pyridin-2-yl)-piperazin-1-yl]-ethyl}-cyclohexyl)-acetamide

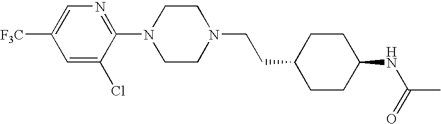

### Step 1: (trans-4-{2-[4-(3-Chloro-5-trifluoromethyl-pyridin-2-yl)-piperazin-1-yl]-ethyl}-cyclohexyl)-carbamic acid tert-butyl ester (Intermediate C)

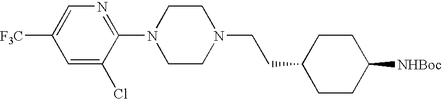

1-(2,3-Dichlorophenyl)-piperazine hydrochloride (1. g, 3.8 mmol) was dissolved in CH₂Cl₂) and [trans-4-(2-oxo-ethyl)-cyclohexyl]-carbamic acid tert-butyl ester (Intermediate A, 908 mg, 3.8 mmol) was added. After 3 h Na(AcO)₃BH (1.44 g, 6.8 mmol) was added and stirring continued over night at 25° C. Sat. aq. NaHCO₃ was added and the product was extracted with 3 portions of CH₂Cl₂. The combined organic layers were dried (MgSO₄) and the solvent was evaporated. Flash chromatography (50 g SiO₂; Hept:EtOAc 80:20->0:100) afforded 1.67 g (90%) of pure title compound as a white solid. m/z: 391.0 ([M+H]⁺).

### Step 2: trans-4-{2-[4-(3-Chloro-5-trifluoromethyl-pyridin-2-yl)-piperazin-1-yl]-ethyl}-cyclohexylamine trihydrochloride (Intermediate D)

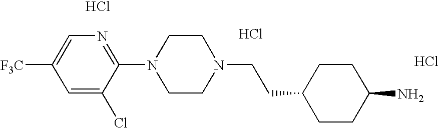

(trans-4-{2-[4-(3-Chloro-5-trifluoromethyl-pyridin-2-yl)-piperazin-1-yl]-ethyl}-cyclohexyl)-carbamic acid tert-butyl ester (1.67 g, 3.4 mmol) was dissolved in CH₂Cl₂ (15 ml). 4 N HCl in dioxane (17 ml, 68 mmol) was slowly added and the resulting mixture was stirred over night at 25° C. ⁱPr₂O (20 ml) was added and the solid product was collected by filtration and it was washed with more ⁱPr₂O (20 ml).

Drying at 50° C. for 1 h on the high vacuum afforded 1.46 g (85%) of the title compound as a white solid. m/z: 391.2 ([M+H]⁺).

### Step 3: N-(trans-4-{2-[4-(3-Chloro-5-trifluoromethyl-pyridin-2-yl)-piperazin-1-yl]-ethyl}-cyclohexyl)-acetamide

A solution of trans-4-{2-[4-(3-chloro-5-trifluoromethyl-pyridin-2-yl)-piperazin-1-yl]-ethyl}-cyclohexylamine trihydrochloride_(150 mg, 0.3 mmol), acetic acid (25 mg, 0.42 mmol), ⁱPr₂NEt (0.18 ml, 1.0 mmol) and TBTU (135 mg, 0.42 mmol) in DMF was stirred 2 h at 25° C. Sat. aq. NaHCO₃ was added and the product was extracted with 3 portions of CH₂Cl₂. The organic phases were combined and passed through a column (20 g SiO₂; EtOAc/MeOH 100:0->80:20) to yield 84 mg (63%) of title compound as a white solid. m/z: 433.2 ([M+H]⁺).

### Examples 2-4

Examples 2-4 were prepared in analogy to example 1 starting from trans-4-{2-[4-(3-chloro-5-trifluoromethyl-pyridin-2-yl)-piperazin-1-yl]-ethyl}-cyclohexylamine trihydrochloride (Intermediate D) and an appropriate carboxylic acid.

TABLE 2
examples 2-4

| | | | m/z |
|---|---|---|---|
| Ex. | Compound | Carboxylic acid | ([M + H]⁺) |
| 2 | N-(trans-4-{2-[4-(3-Chloro-5- | from 3-methoxy- | 477.0 |
| | trifluoromethyl-pyridin-2-yl)- | propionicacid | |
| | piperazin-1-yl]-ethyl}- | | |
| | cyclohexyl)-3-methoxy- | | |
| | propionamide | | |
| 3 | N-(trans-4-{2-[4-(3-Chloro-5- | from propionic acid | 447.3 |
| | trifluoromethyl-pyridin-2-yl)- | | |
| | piperazin-1-yl]-ethyl}- | | |
| | cyclohexyl)-propionamide | | |
| 4 | N-(trans-4-{2-[4-(3-Chloro-5- | from cyclopropylacetic | 473.2 |
| | trifluoromethyl-pyridin-2-yl)- | acid | |
| | piperazin-1-yl]-ethyl}- | | |
| | cyclohexyl)-2-cyclopropyl- | | |
| | acetamide | | |

### Example 5

### N-(trans-4-{2-[4-(3-Chloro-pyridin-2-yl)-piperazin-1-yl]-ethyl}-cyclohexyl)-acetamide

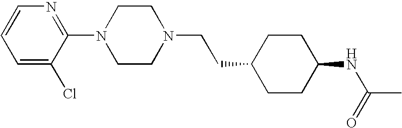

A solution in CH₂Cl₂ (5 ml) of 1-(3-chloro-pyridin-2-yl)-piperazine hydrochloride (50 mg, 0.21 mmol, J. Med. Chem. 2005, 48(6), 1857-1872), N-[trans-4-(2-oxo-ethyl)-cyclohexyl]-acetamide (Intermediate B, 47 mg, 0.26 mmol) Et₃N (26 mg, 0.26 mmol) and Na(AcO)₃BH (81 mg, 0.38 mmol) was stirred 3 h at 25° C. Sat. aq. NaHCO₃ was added and the product was extracted with CH₂Cl₂ (2×20 ml). The combined organic layers were dried (Na₂SO₄) and the solvent was evaporated. Flash chromatography (10 g SiO₂; CH₂Cl₂:MeOH 100:0->85:15) afforded 42 mg (54%) of the title compound as a white solid. m/z: 365.3 ([M+H]⁺).

### Example 6

### N-(trans-4-{2-[4-(3,5-Dichloro-pyridin-2-yl)-piperazin-1-yl]-ethyl}-cyclohexyl)-acetamide

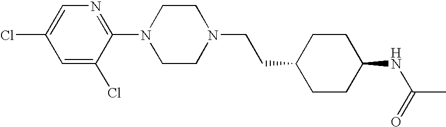

The title compound was prepared in analogy to Example 5 starting from 1-(3,5-dichloro-pyridin-2-yl)-piperazine. No Et₃N was used for this reaction. White solid. m/z: 399.2 ([M+H]⁺).

### Example 7

### N-(trans-4-{2-[4-(6-Trifluoromethyl-pyridin-3-yl)-piperazin-1-yl]-ethyl}-cyclohexyl)-acetamide

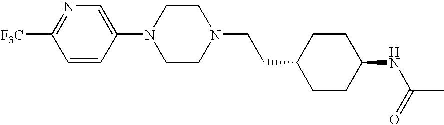

The title compound was prepared in analogy to Example 5 starting from 1-(6-trifluoromethyl-pyridin-3-yl)-piperazine (WO2005014563(A1)). No Et₃N was used for this reaction. White solid. m/z: 399.2 ([M+H]⁺).

### Example 8

### N-(trans-4-{2-[4-(3-Chloro-5-trifluoromethyl-pyridin-2-yl)-piperazin-1-yl]-ethyl}-cyclohexyl)-malonamide

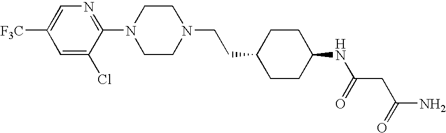

Methyl malonate monoamide (42 mg, 0.36 mmol) was dissolved in CH₂Cl₂ (2 ml) and potassiumtrimethylsilanolate (66 mg, 0.51 mmol) was added. The reaction mixture was stirred 3 h at 25° C., then the solvent was evaporated. The residue was dissolved in dioxane (5 ml) and trans-4-{2-[4-(3-chloro-5-trifluoromethyl-pyridin-2-yl)-piperazin-1-yl]-ethyl}-cyclohexylamine trihydrochloride (100 mg, 0.20 mmol), ⁱPr₂NEt (0.17 ml, 1.0) and TBTU (99 mg, 0.31 mmol) were added. After stirring 2 h at 25° C. the solvent was evaporated, sat. aq. NaHCO₃ was added and the product was extracted with 2 portions of CH₂Cl₂. The organic phases were combined, dried (Na₂SO₄) and the solvent evaporated. Flash chromatography (20 g SiO₂; CH₂Cl₂/MeOH 100:0->80:20) yielded 17 mg (18%) of the title compound as a white solid. m/z: 476.2 ([M+H]⁺).

### Example 9

### N-(trans-4-{2-[4-(3-Methoxy-pyridin-2-yl)-piperazin-1-yl]-ethyl}-cyclohexyl)-acetamide

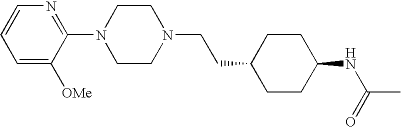

The title compound was prepared in analogy to Example 5 from 1-(3-methoxy-pyridin-2-yl)-piperazin dihydrochloride. Off-white solid. m/z: 361.2 ([M+H]⁺).

### Example 10

### N-(trans-4-{2-[4-(2,3-Dichloro-pyridin-4-yl)-piperazin-1-yl]-ethyl}-cyclohexyl)-acetamide

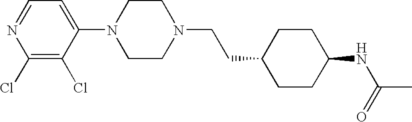

The title compound was prepared in analogy to Example 5 from 1-(2,3-dichloro-pyridin-4-yl)-piperazine hydrochloride (Intermediate E3). Off-white solid. m/z: 399.2 ([M+H]⁺).

### Synthesis of Intermediates

### Intermediate A

### [trans-4-(2-oxo-ethyl)-cyclohexyl]-carbamic acid tert-butyl ester

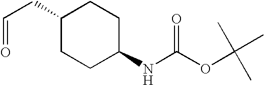

The title compound was prepared as described in WO2007/093540.

### Intermediate B

### N-[trans-4-(2-oxo-ethyl)-cyclohexyl]-acetamide

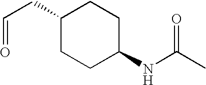

The title compound was prepared as described in WO2007/093540.

### Intermediate E1

### 4-(2-Chloro-pyridin-4-yl)-piperazine-1-carboxylic acid tert-butyl ester

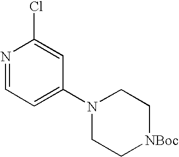

2,4-Dichloropyridine (1.00 g, 6.7 mmol) and piperazine-1-carboxylic acid tert-butyl ester (1.64 g, 8.8 mmol) were suspended in DMF (10 ml) and ⁱPr₂NEt (2.30 ml, 14 mmol) was added. After stirring over night at 120° C. the reaction mixture was diluted with H₂O and extracted with EtOAc. The organic layer was dried (Na₂SO₄) and the solvent was evaporated. The residue was purified by flash chromatography (SiO₂ 50 g, nHept/EtOAc 5 to 100%) to yield 1.02 g (51%) of product and 450 mg (22%) of the regioisomer as byproduct. Light yellow solid. m/z: 298.4 ([M+H]⁺).

### Intermediate E2

### 4-(2,3-Dichloro-pyridin-4-yl)-piperazine-1-carboxylic acid tert-butyl ester

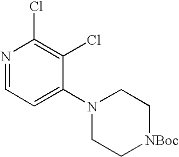

A stirred solution of 4-(2-chloro-pyridin-4-yl)-piperazine-1-carboxylic acid tert-butyl ester (900 mg, 3.0 mmol) in CHCl₃ (20 ml) was treated with AcOH (4 ml) and N-chlorosuccinimide (605 mg, 4.5 mmol). The reaction mixture was stirred 6 h under reflux, the it was poured on ice and the pH was raised to 7 by addition of solid NaHCO₃. The product was extracted with 2 portions of CH₂Cl₂. After drying (Na₂SO₄) and evaporation of the solvent, the residue was purified by flash chromatography (SiO₂ 50 g, nHept/EtOAc 5 to 100%) to yield 400 mg (40%) of title compound as white solid. m/z: 332.2/334.3 ([M+H]⁺).

### Intermediate E3

### 1-(2,3-Dichloro-pyridin-4-yl)-piperazine hydrochloride

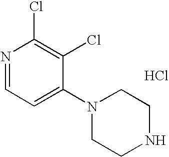

4-(2,3-Dichloro-pyridin-4-yl)-piperazine-1-carboxylic acid tert-butyl ester (380 mg, 1.1 mmol) was dissolved in CH₂Cl₂ (5 ml). 4 N HCl in dioxane (5.72 ml, 23 mmol) was added and the resulting mixture was stirred 5 h at 25° C. ⁱPr₂O (10 ml) was added and the solid product was collected by filtration. Drying on the high vacuum finally yielded 350 mg (quant.) of the title compound as white solid. m/z: 232.2/234.1 ([M+H]⁺).

### Pharmaceutical Preparations

### Example A

Film coated tablets containing the following ingredients can be manufactured in a conventional manner:

TABLE 3
Example of film coated tablets

| Ingredients | Per tablet | |
|---|---|---|
| Kernel: | | |
| Compound of formula I | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titanium dioxide | 0.8 mg | 1.6 mg |

The active ingredient is sieved and mixed with microcrystalline cellulose and the mixture is granulated with a solution of polyvinylpyrrolidone in water. The granulate is mixed with sodium starch glycolate and magnesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

TABLE 4
Example of capsules

| Ingredients | Per capsule |
|---|---|
| Compound of formula I | 25.0 mg |
| Lactose | 150.0 mg |
| Maize starch | 20.0 mg |
| Talc | 5.0 mg |

The components are sieved and mixed and filled into capsules of size 2 or other suitable sizes.

### Example C

Injection solutions can have the following composition:

TABLE 5
Example of injection solutions

| Compound of formula I | 3.0 | mg |
|---|---|---|
| Gelatine | 150.0 | mg |
| Phenol | 4.7 | mg |
| Sodium carbonate | to obtain a final pH of 7 | |
| Water for injection solutions | ad 1.0 | ml |

### Example D

Soft gelatin capsules containing the following ingredients can be manufactured in a conventional manner:

TABLE 6
Example of soft gelatin capsules

| Capsule contents | | |
|---|---|---|
| Compound of formula I | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titanium dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

TABLE 7
Example of sachets

| | | |
|---|---|---|
| Compound of formula I | 50.0 | mg |
| Lactose, fine powder | 1015.0 | mg |
| Microcrystalline cellulose (AVICEL PH 102) | 1400.0 | mg |
| Sodium carboxymethyl cellulose | 14.0 | mg |
| Polyvinylpyrrolidone K 30 | 10.0 | mg |
| Magnesium stearate | 10.0 | mg |
| Flavoring additives | 1.0 | mg |

The active ingredient is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

1. A compound of formula I: 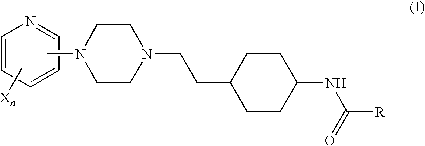
wherein:
X is independently halogen, C₁₋₆-alkyl, C₁₋₆-haloalkyl or C₁₋₆-alkoxy;
n is 1 or 2;
R is C₁₋₆-alkyl, wherein C₁₋₆-alkyl is optionally substituted by —CONH₂, C₁₋₆-alkoxy, or one 3 to 6 membered monocyclic cycloalkyl; or
C₁₋₆-alkoxy;
or a pharmaceutically acceptable salt thereof.

2. The compound of claim 1, wherein X is independently chlorine, fluorine, —CF₃ or —OCH₃.

3. The compound of claim 1, wherein X is halogen.

4. The compound of claim 3, wherein X is fluorine.

5. The compound of claim 3, wherein X is chlorine.

6. The compound of claim 1, wherein X is C₁₋₆-alkyl.

7. The compound of claim 1, wherein X is C₁₋₆-haloalkyl.

8. The compound of claim 7, wherein X is CF₃.

9. The compound of claim 1, wherein X is C₁₋₆-alkoxy.

10. The compound of claim 9, wherein X is OCH₃.

11. The compound of claim 1, wherein n is 1.

12. The compound of claim 1, wherein n is 2.

13. The compound of claim 1, having formula I′:

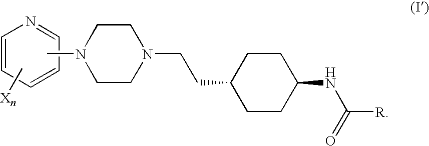

14. The compound of claim 1, having formulae Ia or Ia′:

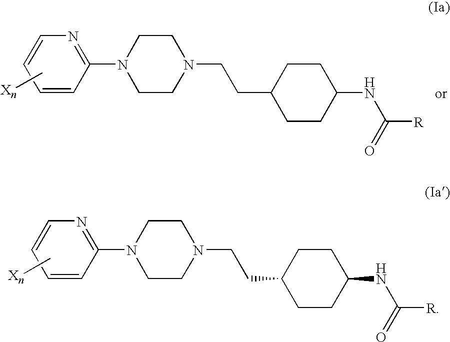

15. The compound of claim 1, having formulae Ib or Ib′: 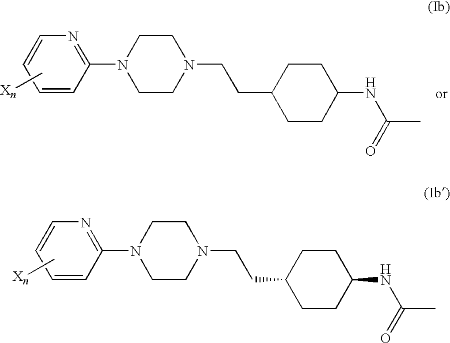
wherein:
X is independently fluorine, chlorine, —CF₃ or —OCH₃; and
n is 1 or 2.

16. The compound of claim 15, selected from the group consisting of:
N-(trans-4-{2-[4-(3-Chloro-5-trifluoromethyl-pyridin-2-yl)-piperazin-1-yl]-ethyl}-cyclohexyl)-acetamide;
N-(trans-4-{2-[4-(3-Chloro-5-trifluoromethyl-pyridin-2-yl)-piperazin-1-yl]-ethyl}-cyclohexyl)-3-methoxy-propionamide;
N-(trans-4-{2-[4-(3-Chloro-5-trifluoromethyl-pyridin-2-yl)-piperazin-1-yl]-ethyl}-cyclohexyl)-propionamide;
N-(trans-4-{2-[4-(3-Chloro-5-trifluoromethyl-pyridin-2-yl)-piperazin-1-yl]-ethyl}-cyclohexyl)-2-cyclopropyl-acetamide;
N-(trans-4-{2-[4-(3-Chloro-pyridin-2-yl)-piperazin-1-yl]-ethyl}-cyclohexyl)-acetamide;
N-(trans-4-{2-[4-(3,5-Dichloro-pyridin-2-yl)-piperazin-1-yl]-ethyl}-cyclohexyl)-acetamide;
N-(trans-4-{2-[4-(6-Trifluoromethyl-pyridin-3-yl)-piperazin-1-yl]-ethyl}-cyclohexyl)-acetamide;
N-(trans-4-{2-[4-(3-Chloro-5-trifluoromethyl-pyridin-2-yl)-piperazin-1-yl]-ethyl}-cyclohexyl)-malonamide;
N-(trans-4-{2-[4-(3-Methoxy-pyridin-2-yl)-piperazin-1-yl]-ethyl}-cyclohexyl)-acetamide; and
N-(trans-4-{2-[4-(2,3-Dichloro-pyridin-4-yl)-piperazin-1-yl]-ethyl}-cyclohexyl)-acetamide.

17. The compound of claim 1, wherein R is methyl, methyl substituted by —CONH₂, methyl substituted by cyclopropyl, ethyl or ethyl-OCH₃.

18. The compound of claim 1, wherein R is C₁₋₆-alkoxy.

19. The compound of claim 1, wherein R is a 3 to 6 membered monocyclic cycloalkyl.

20. The compound of claim 1, wherein R is C₁₋₆-alkyl optionally substituted by CONH₂, C₁₋₆-alkoxy, or one 3 to 6 membered monocyclic cycloalkyl.

21. The compound of claim 20, wherein R is C₁₋₆-alkyl.

22. The compound of claim 21, wherein R is methyl.

23. The compound of claim 21, wherein R is ethyl.

24. The compound of claim 20, wherein R is C₁₋₆alkyl substituted by CONH₂.

25. The compound of claim 24, wherein R is methyl substituted by CONH₂.

26. The compound of claim 20, wherein R is C₁₋₆-alkyl substituted by a 3 to 6 membered monocyclic cycloalkyl.

27. The compound of claim 26, wherein R is cyclopropyl-methyl.

28. The compound of claim 20, wherein R is C₁₋₆-alkyl substituted by C₁₋₆-alkoxy.

29. The compound of claim 28, wherein R is methoxy-ethyl.

30. A pharmaceutical composition comprising a therapeutically effective amount of a compound of formula I 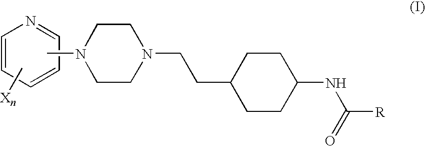
wherein:
X is independently halogen, C₁₋₆-alkyl, C₁₋₆-haloalkyl or C₁₋₆-alkoxy;
n is 1 or 2;
R is C₁₋₆-alkyl, wherein C₁₋₆-alkyl is optionally substituted by —CONH₂, C₁₋₆-alkoxy, or one 3 to 6 membered monocyclic cycloalkyl; or
C₁₋₆-alkoxy;
or a pharmaceutically acceptable salt thereof and a pharmaceutically acceptable carrier.
